(19) United States
(12) Patent Application Publication
(10) Pub. No.: US 2010/0323984 A1
(43) Pub. Date: Dec. 23, 2010

(54) COSMETIC ORAL AND/OR PARENTERAL USE OF GLUCOSAMINE OPTIONALLY IN COMBINATION WITH AT LEAST ONE POLYPHENOL COMPOUND, AND CORRESPONDING COMPOSITION

(75) Inventors: Antoine Piccirilli, Poitiers (FR); Patricia Manissier, Levallois-Perret (FR); Christiane Montastier, Paris (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: LABORATOIRES INNEOV, Asnieres Cedex (FR)

(21) Appl. No.: 12/445,960

(22) PCT Filed: Oct. 19, 2007
(86) PCT No.: PCT/FR07/52202

§ 371 Date: Jul. 14, 2009

(30) Foreign Application Priority Data
Oct. 20, 2006 (FR) 0654425
Oct. 20, 2006 (FR) 0654426

(51) Int. Cl. A61K 31/7008; A61K 8/60; A61K 31/715; A61P 17/00; A61Q 19/00; A61Q 19/08; A61Q 19/06; A61P 17/02

(52) U.S. Cl. 514/54; 514/62

(57) ABSTRACT

The present invention relates to the cosmetic oral and/or parenteral use of glucosamine as an active agent for preventing and/or treating the cutaneous signs of ageing, optionally in combination with at least one polyphenol compound.

The present invention also relates to a composition for oral and/or parenteral administration comprising, as active agent, the combination of glucosamine and of at least one polyphenol compound derived from pine bark.

The present invention relates to the field of dietary supplements or functional food products intended for skin care.

Human skin is constituted of three compartments, namely a superficial compartment, the epidermis, the dermis and a deep compartment, the hypodermis. The latter compartment is essentially constituted of one type of cells specialized in fat accumulation and storage, adipocytes. The hypodermis is the organism's energy reservoir.

Natural human epidermis is composed mainly of three types of cells, namely keratinocytes, which form the vast majority, melanocytes and Langerhans cells. Each of these cell types contributes, via its intrinsic functions, to the essential role played by the skin.

The dermis provides the epidermis with a solid support. It is also its feeder element. It is mainly constituted of fibroblasts and of an extracellular matrix which is itself composed mainly of collagen, elastin and a substance known as ground substance, these components being synthesized by the fibroblasts.

Leukocytes, mast cells or tissue macrophages are also found therein. The dermis is also interlaced with blood vessels and nerve fibers. In normal skin, i.e. skin that is neither pathological nor cicatricial, the fibroblasts are in the quiescent state, i.e. nonproliferative state.

The solidity of the dermis is provided by the collagen fibers. The collagen fibers are constituted of fibrils sealed together, thus forming more than ten different types of structures. The solidity of the dermis is largely due to the entanglement of the collagen fibers, which are packed tightly together in all directions. The collagen fibers contribute to the elasticity and to the tonicity of the skin and/or of mucous membranes.

The collagen fibers are constantly replaced, but this replacement decreases with age, thereby resulting in a thinning of dermis. This thinning of the dermis is also due to pathological causes, such as, for example, hypersecretion of corticoid hormones, certain pathological conditions or else vitamin deficiencies (in the case of vitamin C in scurvy). It is also accepted that extrinsic factors such as ultraviolet rays, tobacco or some treatments (glucocorticoids, vitamin D and derivatives, for example) also have an effect on the skin and on its level of collagen.

Various factors lead to collagen degradation, with all the consequences that can be envisioned with regard to the structure and/or the firmness of the skin and/or mucous membranes.

Although highly resistant, collagen fibers are sensitive to certain enzymes known as collagenases. Degradation of the collagen fibers results in the appearance of flabby and wrinkled skin which human beings, preferring the appearance of a smooth and taut skin, have always sought to combat.

Collagenases belong to a family of enzymes known as metalloproteinases (MMPs) which are themselves members of a family of proteolytic enzymes (endoproteases or endopeptidases) which have a zinc atom coordinated to 3 cysteine residues and a methionine in their active site and which degrade the macromolecular components of the extracellular matrix and of the basal laminae at neutral pH (collagen, elastin, etc.). Very wide spread in the living world, these enzymes are present, but weakly express, in normal physiological situations such as organ growth and tissue replacement.

Their overexpression in humans and their activation are related to many processes, sometimes pathological processes, which involve the destruction and remodeling of the matrix. This leads either to uncontrolled resorption of the extracellular matrix or, conversely, to a state of fibrosis setting in.

The metalloproteinase family is constituted of several well-defined groups based on their similarities in terms of structure and of substrate specificity. Among these groups, mention may be made of collagenases intended to degrade fibrillar collagens (MMP-1 or interstitial collagenase, MMP-8 or neutrophil collagenase, MMP-13 or collagenase 3), gelatinases which degrade collagen type IV or any form of denatured collagen (MMP-2 or gelatinase A (72 kDa), MMP-9 or gelatinase B (92 kDa)).

Stromelysins (MMP-3) have, for their part, a broad spectrum of activity which applies to proteins of the extracellular matrix, such as glycoproteins (fibronectin, laminin), proteoglycans, etc., or else membrane metalloproteinases. The latter do not have the anti-collagenase role of the metalloproteinases reported above.

In addition, certain proteoglycans such as those which belong to the small leucine-rich proteoglycan (SLRP) family constitute an advantageous target with a view to preventing the negative effects of ageing and the deterioration of the mechanical properties of the skin. These SLRPs are in fact directly involved in fibrillogensis and the hydration of perifibrillar spaces. SLRPs contribute in particular to increasing the bioavailability of certain growth factors such as TGF-β: among the SLRPs, mention may be made of decorin, lumican, fibromodulin and biglycan. Moreover, certain immunohistochemical observations demonstrate a decrease in the accumulation of biglycan in aged skin. Similarly, the marked decrease in lumican and in fibromodulin induces an impaired collagen fibrillogensis and also a disturbed fibrillar architecture. Consequently, the proteoglycans of the SLRP family play a fundamental role in the architectural organization of the structures of the skin and therefore in the regulation of skin firmness.

Finally, SLRPs are not only sensitive to the action of MMPs, but also to the proteolytic action of aggrecanases or ADAMTS (A Disintegrin And Metalloprotease with Thrombospondin type I repeat). Certain members of this new metalloprotease family, in particular ADAMTS 1 and 4, have been identified in the skin, and ADAMTS4 is known to cleave decorin.

Prolonged exposure to ultraviolet radiation, particularly to type A or B ultraviolet radiation, has the effect of stimulating the expression of collagenases, in particular of MMP-1. This is one of the components of photoinduced cutaneous ageing.

Furthermore, at the menopause, the main modifications relating to the dermis are a decrease in the level of collagen and in the dermal thickness. This results in thinning of the skin and/or of the mucous membranes in menopausal woman. Women then experience a “wizened skin” or tight skin feeling and an accentuation of surface fine lines and wrinkles is observed. The skin has a rough feeling on palpation. Finally, the skin exhibits reduced suppleness.

Finally, in overweight individuals, and more particularly during weight gain, the adipocytes have a tendency to rapidly increase in volume (storage of increasing amounts of lipids). The fat lobules then distend little by little so as to result in the formation of connective trabeculae, parallel to one another and perpendicular to the skin surface. The strong pressure exerted by the adipocytes on the dermis rapidly causes a deformation of the surface of the skin. In cutaneous terms, this “cellulite” phenomenon is reflected by a padded appearance giving, in places, the signs of “orange peel”. Finally, from the clinical point of view, cellulite is reflected by a modification of the texture of the subcutaneous and superficial tissues, characterized, in particular, by:
skin which, overall, is thicker,
skin which is more consistent,
skin which is more sensitive, and which can, depending on the stage of progression of the cellulite, be painful upon palpation, and/or
cutaneous tissues which are less mobile due to the loss of adhesion and of cohesion of the deep layers of the skin.

Thus, this phenomenon is more visible in women since they have finer skin with connective trabeculae exhibiting a vertical structure, which, on the other hand in men, have an oblique and criss-cross structure.

Cellulite, which is often worsened by excess weight and obesity, is especially located around the hips and the lower limbs (“jodhpur thigh” or “zouave pants” cellulite). These modifications can thus result in permanent scarring deformations.

Hypertrophy of the adipose tissue is accompanied, at the dermal level, by the fiber network being placed under tension, leading to a functional impairment of the resident cells. In fact, this hypertension impedes cellular exchanges and venous and lymphatic circulation by compression of capillaries, to such an extent that the phenomenon is self-maintaining. In the end, the fibers degenerate and the skin loses its fundamental structures.

From the biological point of view, when the fibroblasts are subjected to a normal tissue tension, they actively synthesise collagen, elastin and glycoaminoglycans, which are essential molecules that contribute to reinforcing the supporting tissues of the skin. Similarly, adipocytes overloaded with lipids also exert a tension on the dermis, leading to overproduction of collagen until fibrosis occurs. This is reflected, in clinical terms, by skin which is more consistent and taut.

On the other hand, during weight loss, and in particular during slimming diets, the rapid destorage of the adipocytes leads to a considerable decrease in the tension exerted by the hypodermis on the supporting tissues. Consequently, since the dermis is no longer under tension, the connective tissue gradually loses its cohesion: loss of attachment of the fibroblasts to the collagen, decrease in the amount of neocollagen, distension of elastin fibers, depolymerization of proteoglycans, etc. Consequently, the fibroblasts, which have less interaction with the fibers of the extracellular matrix, no longer receive from their environment the activity and repair signals which control the synthesis of the essential macromolecules of the dermis. In addition, the fibroblasts which are no longer receiving signals from their fibrillar environment secrete matrix metalloproteases (MMPs), enzymes that result in degradation of the fibrous structures. This marked slowing of the fibroblast metabolism, and also the degradation of the fibers by the MMPs, is consequently reflected by an impairment of the viscoelastic or biomechanical properties of the skin (loss of firmness, of tonicity, of elasticity, etc.).

On reading the above, it is then possible to understand the importance of collagen and glycosaminoglycans in the structure of tissues, in particular of the skin and/or the mucous membranes, and the importance in combating degradation thereof in order to thus combat ageing, whether it is chronobiological or photoinduced ageing, and the consequences thereof, in particular on thinning of the dermis and/or degradation of collagen fibers, the latter consequence leading to a loss of skin firmness and, in particular, the appearance of flabby skin, the object of the present invention being precisely to combat this.

The present invention focuses more particularly on the prevention and/or treatment, by oral and/or parenteral administration, of the cutaneous signs of ageing, and most particularly of the cutaneous signs associated with an impairment of the viscoelastic or biomechanical properties of the skin, especially loss of skin firmness, and also on the prevention and/or treatment of cellulite.

It is noted that, in the literature, one of the approaches described for combating cellulite consists in stimulating lipolysis, for example by inhibiting phosphodiesterase or by activating β-adrenoreceptors.

The oral administration of glucosamine is principally known in the treatment of arthrosis, in particular in the form of a dietary supplement.

The document “The effect of an oral supplement containing glucosamine, amino acids, minerals, and antioxidants on cutaneous ageing: a preliminary study” [symbol] , H. MURAD and Michael TABIBIAN, Journal of Dermatological Treatment (2001)12, 47-51 reports a study on a group of volunteers treated orally with a product containing multiple ingredients, namely acetylated D-glucosamine, glucosamine sulfate, L-proline, L-lysine, quercetin zinc, copper, manganese and a grapeseed extract. The results appear to show an effect on the decreased number of wrinkles, observed after treatment.

The topical use of acetylglucosamine is, moreover, known as a skin conditioner. Acetylglucosamine is also an ingredient in certain creams, in particular moisturizing creams, for improving the appearance of the skin.

The document EP 1 075 836 describes a skin care composition comprising N-acetylglucosamine as active agent.

The document JP 2004-083432 describes an elastase inhibitor for improving wrinkles and skin ageing, comprising glucosamine, derivatives thereof or salts thereof.

Finally, the document JP 2004-083434 describes an agent for promoting collagen synthesis, comprising glucosamine, derivatives thereof or salts thereof.

The subject of the invention is the cosmetic oral and/or parenteral use of glucosamine as active agent for preventing and/or treating skin disorders induced by cellulite and/or for maintaining and/or restoring skin firmness.

A subject of the invention is also the use of glucosamine for the preparation of a pharmaceutical composition for oral and/or parenteral administration for preventing and/or treating the cutaneous signs of ageing, and in particular skin disorders associated with cutaneous atrophy and/or with deteriorated collagen synthesis and/or with overexpression of MMP3.

Topical treatments for combating the cutaneous signs of ageing are known. However, the topical active agents recommended do not always act, due to their weak cutaneous penetration, at the dermal level. In addition, the topical products act, by definition, locally on the areas to be treated, areas on which they may be unequally distributed, and require careful and repeated applications. In some cases, they may be responsible for cutaneous side reactions, or even discomfort.

In contrast, oral and/or parenteral administration has the advantage of acting globally on the entire skin, and in these deep layers (dermis, hypodermis). In fact, the glucosamine and/or metabolites thereof are then distributed within the dermal matrix by means of the blood stream.

In the context of the present invention, the term “parenteral administration” is understood to mean intramuscular injection, intravenous injection or else administration via a systemic patch. In other words, this definition is intended to cover all the other methods of administration other than oral (or gastrointestinal) as long as the active agents pass through the blood stream.

Administration by means of a systemic patch is preferred as parenteral administration. The patch with exclusively local effect is excluded from the present invention.

Thus, oral administration or administration by patch also have the advantage of a rapid and relatively nonrestricting administration.

In contrast, topical administration, due to the weaker cutaneous penetration, does not always make it possible to use all the properties of the active agents, at the dermal level.

In addition, topical products act, by definition, locally on the areas to be treated, areas on which they may be unequally distributed, and require careful and repeated applications. In certain cases, they may be responsible for cutaneous side reactions, or even discomfort.

The inventors have also more particularly discovered that the oral administration of the combination of glucosamine and at least one polyphenol compound, in particular a polyphenol compound derived from pine bark, has a beneficial activity on the skin. The inventors have also more particularly observed that this combination is advantageous, when it is administered orally, in particular on the maintenance and/or restoration of the biomechanical properties of the skin. It makes it possible even more particularly to maintain and/or restore the extensibility, tonicity, firmness, suppleness and/or elasticity properties of the skin and/or to prevent and/or treat skin disorders induced by cellulite.

Polyphenol compounds are in particular known for their strong antioxidant capacity and are commonly used in cosmetic products. Their role in the prevention of cardiovascular diseases by oral administration has also been described.

The use, in cosmetics, of a maritime pine bark extract, in particular sold under the name Pycnogenol®, is described in the document F. Schonlau, “The cosmeceutical pycnogenol®”, Journal of applied cosmetology, Vol. 20, No. 4, 2002, pages 241-246.

Thus, in the context of the present invention, glucosamine can be combined with at least one polyphenol compound.

The invention therefore also relates to the cosmetic oral use of the combination of glucosamine and of at least one polyphenol compound as a mixture of active agents for maintaining and/or restoring the biomechanical properties of the skin and/or for preventing and/or treating skin disorders induced by cellulite.

The invention also relates to the cosmetic oral and/or parenteral use of the combination of glucosamine and of at least one polyphenol compound as a mixture of active agents for promoting cicatrization.

The invention also relates to the use of a combination of glucosamine and of at least one polyphenol compound, for the preparation of a composition for oral and/or parenteral administration for preventing and/or treating the cutaneous signs of ageing associated with a loss of extensibility, of tonicity, of firmness, of suppleness, of density and/or of elasticity of the skin.

The invention also relates to a composition for oral administration comprising the combination of glucosamine and of at least one polyphenol compound derived from pine bark.

The invention also relates to a dietary supplement or a functional food product comprising glucosamine in a first composition and at least one polyphenol compound derived from pine bark in a second composition, as a kit or combination product for simultaneous, separate or sequential use.

In the context of the present invention, the expression “viscoelastic or biomechanical properties of the skin” is intended to mean the extensibility, tonicity, firmness, suppleness, density and/or elasticity properties of the skin.

The expression “cutaneous signs of ageing” is intended to mean any modifications of the external appearance of the skin due to ageing, whether it is chronobiological and/or extrinsic, in particular photoinduced and/or hormonal, in particular wrinkles and fine lines, wizened skin, flabby skin, thinned skin, dull skin which is not radiant, a lack of skin elasticity and/or tone, but also any internal modifications of the skin which are not systematically reflected by a modified external appearance, for instance any internal damage to the skin, in particular to the collagen fibers, subsequent to exposure to ultraviolet radiation.

This term is considered to be equivalent to the term “skin disorders induced by chronological ageing and/or extrinsic ageing and/or hormonal ageing”.

The expression “skin disorder induced by slimming and/or weight-loss diets” is intended to mean all the modifications of the external appearance of the skin, for instance the flaccid skin appearance that can be more or less marked following weight loss.

In the context of the present invention, the expression “skin disorders induced by cellulite” is intended to mean not only all the modifications of the external appearance of the skin, for instance the nodes of fat or the “orange peel” which can be more or less localized in the areas of excess weight, such as the thighs, the arms or the abdomen, but also the pain on palpation, whereas the expression “visual aspects associated with cellulite” covers all the modifications of the external appearance of the skin, for instance nodes of fat and “orange peel” only.

For the purpose of the invention, the term “patch” or “transdermal device” or “transdermal delivery system” is intended to mean any system allowing active or passive release of the active substance via the transdermal route, i.e. allowing its transfer through the skin to the systemic circulation.

It is understood, in the context of the present invention, that “the cosmetic oral and/or parenteral use” covers the use of products administered orally and/or parenterally, these products, for example in the form of a dietary supplement or of a functional element as disclosed hereinafter for the case of oral administration, producing an effect, on the skin, from the esthetic or comfort point of view, or else for beauty purposes, for example with a view to protecting it, to maintaining it in good condition, to modifying the appearance thereof, and especially to embellishing it.

Glucosamine

Glucosamine is an amino sugar, in particular of marine origin composed of glucose base and an amine function. It is a simple sugar, of low molecular mass, and which is in the form of white, water-soluble crystals of formula (1) or (2).

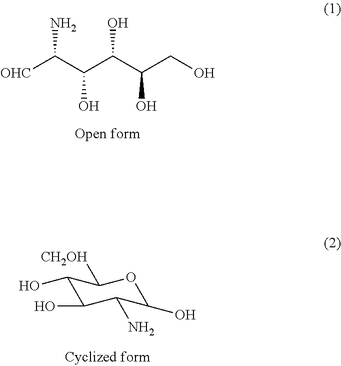

Glucosamine can be obtained from chitin, which is a biodegradable, natural complex sugar that is just as naturally abundant as cellulose.

Chitin is the primary constituent of the shell (exoskeleton) of shellfish such as crabs, shrimps or lobsters. It is therefore a marine polymer composed of glucosamine units. In chitin, more than 60% of the total glucosamine is present in acetylated form.

The chemical formula of chitin is the following:

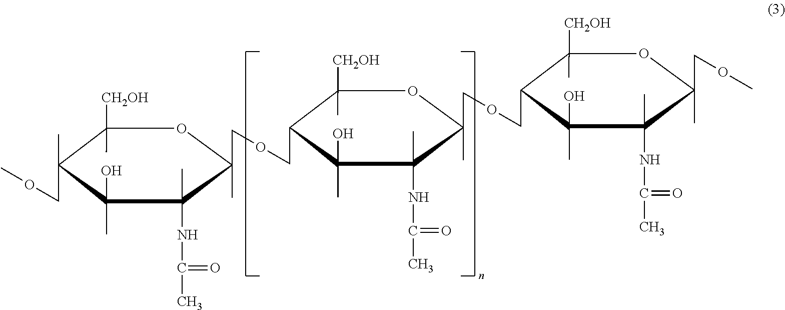

There is another source of chitin that can be used to obtain glucosamine hydrochloride. This is chitin obtained from the biomass produced by fermentation of the fungus A. niger of the class Deuteromycetes, order Monoliales, family Moniliaceae, genus Aspergillus and species niger.

Commercially, glucosamine can be provided in various salt forms: sulfate, sulfate potassium chloride (2 KCl), sulfate sodium chloride (2 NaCl), hydrochloride (HCl), acetylated or else polymerized (N-acetylglucosamine). The nature of the salt often depends on the method of production used. However, the most widely used and studied forms, glucosamine sulfate and glucosamine hydrochloride, correspond to the most soluble salts.

Thus, in the context of the present invention, it is understood that the term “glucosamine” comprises all its salified, acetylated and/or polymeric forms. Among the salified forms of glucosamine, mention may be made of glucosamine sulfate, glucosamine sulfate potassium chloride, glucosamine sulfate sodium chloride and glucosamine hydrochloride.

An example of a production method is reported hereinafter, by way of illustration.

Glucosamine in sulfated form can, for example, be prepared from the shells of shrimps (rich in chitin).

In this method of production, the first step comprises an acid hydrolysis in the presence of hydrochloric acid, carried out under vacuum, at 95° C. The hydrolysis conditions are dependent on the starting material.

Still in this method of production, the reaction medium is subsequently decolored with active carbon, which retains, in passing, the absorbable organic impurities.

After gradual concentration under vacuum, the glucosamine hydrochloride obtained after hydrolysis crystallizes slowly in the cold for several hours. After filtration, the latter is washed with alcohol.

Finally, the last step of this method of production comprises neutralizing the glucosamine hydrochloride with potassium sulfate in the aqueous phase. The mixture is subsequently evaporated and the solvent obtained is dried under vacuum at 60° C. for several hours.

When chitin derived from the abovementioned fungus is used in place of the chitin derived from shrimp shells, said chitin also undergoes hydrolysis so as to produce glucosamine.

The composition according to the invention preferably provides the glucosamine compound in a daily dose ranging from 50 mg to 3 g/day, preferably from 200 to 2000 mg/day, and even more preferably from 250 to 1500 mg/day.

Preferably, the glucosamine is present in the composition of the invention at a content ranging from 0.0001% to 80% by weight, preferably from 1% to 50%, and even more preferably from 10% to 20% by weight, relative to the total weight of the composition.

As illustrated in example 1 which follows, the inventors have demonstrated that glucosamine stimulates collagen synthesis and also the expression of the CD44 hyaluronic acid receptor.

In particular, in this respect, the cosmetic oral and/or parenteral use of glucosamine as fibroblast metabolism activator or collagen synthesis activator and as promoter for reestablishing epidermal homeostasis is also part of the invention.

Example 2 illustrates, in addition, an activity of glucosamine sulfate against the degradation of the dermal matrix constituents, in that it reduces the level of expression of MMP3.

In particular, as a result, the present invention is also directed toward the cosmetic oral and/or parenteral use of glucosamine as an agent for inhibiting the expression of MMP3.

In addition, Example 3 demonstrates a stimulatory effect of glucosamine on the fibroblast cytoskeleton, in that it makes it possible to improve the contractile properties of fibroblasts, a factor which affects skin firmness.

The invention is also directed toward the cosmetic oral and/or parenteral use as an agent for promoting fibroblast contractility.

Finally, Example 4 shows the effect of glucosamine sulfate on the expression of dermal cutaneous markers associated with skin firmness.

The present invention is also directed toward the cosmetic oral and/or parenteral use, as an agent for stimulating the expression of cutaneous markers in particular chosen from vimentin, decorin, fibromodulin, biglycan and hyaluron synthesis.

All these tests particularly underline that glucosamine makes it possible to act on the cellular metabolism and the biomechanical properties of the skin, and mostly on skin firmness.

The invention is therefore also directed toward a cosmetic process for promoting the reestablishment of epidermal homeostasis and/or for limiting degradation of the dermal matrix constituents and/or for reducing the level of expression of MMP3 and/or for stimulating the fibroblast cytoskeleton and/or for improving the contractile properties of fibroblasts and/or for increasing the expression of cutaneous markers in particular chosen from vimentin, decorin, fibromodulin, biglycan and hyaluron synthesis and thus reducing cellulite and also the associated visual aspects and/or maintaining and/or restoring skin firmness, said process comprising the oral and/or parenteral administration of a composition containing glucosamine optionally in combination with a polyphenol compound.

According to one particular embodiment, the invention relates more particularly to the cosmetic oral and/or parenteral use of glucosamine as an active agent for preventing and/or treating skin disorders induced by cellulite and/or for maintaining and/or restoring skin firmness through the action of synthesizing and/or protecting glucosaminoglycans and proteoglycans, and more particularly small-leucine-rich proteoglycans (SLRPs).

The present invention also relates to the cosmetic oral and/or parenteral use of glucosamine as an active agent for maintaining and/or restoring the biomechanical properties of the skin, in particular for maintaining and/or restoring the extensibility, tonicity, firmness, suppleness, density and/or elasticity properties of the skin.

These disorders associated with a loss of the extensibility, tonicity, firmness, suppleness and/or elasticity properties of the skin can in particular be induced by chronological ageing, extrinsic ageing, in particular photoageing, or hormonal ageing, especially of the mature skin of premenopausal or postmenopausal women.

The use of glucosamine orally and/or parenterally can therefore also be advantageously suitable for the prevention and/or treatment of skin disorders, in particular the loss of skin firmness, induced by the menopause.

The oral and/or parenteral use of glucosamine is also particularly suitable for the prevention and/or treatment of the abovementioned skin disorders, i.e. associated with a loss in terms of biomechanical properties of the skin, especially a loss of skin firmness, induced by weight loss, as observed during slimming and weight-loss diets, such as sagging of the supporting tissues, and loss of skin tonicity and elasticity.

The present invention thus also relates to the cosmetic use of glucosamine for preventing and/or combating skin disorders, in particular a loss of skin firmness, induced by weight loss.

This weight loss can be observed, as has been recalled above, during slimming diets. Taking glucosamine orally and/or parenterally thus also makes it possible, in particular in women who are carrying excess weight and are on a slimming diet, to obtain an improvement in skin tonicity and in the flaccid appearance of the skin.

The oral and/or parenteral use of glucosamine can thus be an aid for decongesting the tissues in this context of slimming or weight-loss diets.

The oral and/or parenteral use of glucosamine is, finally, suitable for preventing and/or combating skin disorders induced by cellulite, in particular for the prevention and/or treatment of the visual aspects associated with cellulite, such as nodes of fat and “orange peel skin”, as described above. This is because taking glucosamine orally and/or parenterally makes it possible to improve, in women with cellulite, at a not very or very advanced stage, the orange peel appearance (nodes of fat) observed on skin with cellulite, in particular on the thighs, but also to reduce, or even prevent, the flaccid skin appearance commonly observed, in particular on the arms and the abdomen. Taking glucosamine orally also makes it possible to reduce the pain on palpation of skin with cellulite.

The invention therefore extends to the cosmetic oral and/or parenteral use of glucosamine as an active agent for preventing and/or combating the cutaneous pain or pinching induced by cellulite.

Thus, the present invention also extends to the cosmetic use of glucosamine for preventing and/or treating the visual aspects associated with cellulite, such as nodes of fat and “orange peel appearance”.

In addition, the invention extends to the cosmetic oral and/or parenteral use of glucosamine as an active agent for promoting cicatrization.

Finally, the invention relates to the use of glucosamine for the preparation of a composition for oral and/or parenteral administration for preventing and/or treating the cutaneous signs of ageing, in particular for maintaining and/or restoring the biomechanical properties of the skin, and more particularly preventing and/or treating skin disorders associated with cutaneous atrophy and/or with deteriorated collagen synthesis and/or with overexpression of MMP3.

According to one embodiment of the present invention, the glucosamine can be combined with at least one polyphenol compound.

Polyphenol Compound

The polyphenol compounds group together a large family of compounds that are very widespread in the plant kingdom. They are thus found in particular in plants, from the roots to the fruit. Among the classes of polyphenols, mention may in particular be made of flavonoids, proantocyanidins, lignans, lignins, stilbenes and coumarins. Thus, the polyphenol compound used in the context of the present invention may be in an isolated form or in any of the forms mentioned hereinafter.

In the context of the present invention, the polyphenol compound may in particular derive from plant extracts chosen from extracts of green tea, of grape, such as Vitis vinifera, of pine and in particular of pine bark, of apple, of blueberry, of hops, of guava, of cocoa and of wood, such as chestnut, oak, horse chestnut, hazel.

In the context of the present invention, the term “polyphenol compound” therefore also extends to the plant extract itself, rich in these polyphenol compounds.

Flavonoids represent the main group of polyphenols.

Catechin polyphenols constitute, for their part, a subgroup of flavonoids, which also comprise flavanones, flavones and anthocyanins, and flavonols.

The polyphenol compound present in the composition as first subject of the present invention and in the dietary supplement as second subject of the present invention is derived from pine bark.

Such a polyphenol compound derived from pine bark advantageously has a phenolic trimer content that can range from 5% to 25% by weight, preferably from 10% to 20% by weight, relative to the total weight of the polyphenol content. Similarly, it advantageously has a polyphenolic dimer content that can range from 5% to 25% by weight, preferably from 10% to 20% by weight, relative to the total weight of the polyphenol compound.

The polyphenol compound derived from pine bark also advantageously contains from 2% to 15% by weight, for example from 5% to 10% by weight, of phenolic acids of ferrulic acid, p-coumaric acid, caffeic acid and protocatechic acid type, relative to the total weight of the polyphenol compound.

Thus, the polyphenol compound, which is particularly advantageous for the implementation of the invention, may have the following characteristics:

| Analysis/criterion | Specification |
|---|---|
| Loss on desiccation | ≦5.0% |
| Sulfuric ash | ≦0.4% |
| Insoluble materials in water (solution at 1%, | ≦5.0% |
| T = 37° C.) | |
| Insoluble materials in THF (solution at 1%, | ≦1.0% |
| T = 20° C.) | |
| pH (aqueous solution at 4%, T = 20° C.) | 2.5-4.5 |
| Polyphenolic trimers | 10-20% |
| Polyphenolic dimers | 10-20% |
| Taxifoliol + taxifoliol glucoside | >3% |
| Contents of phenolic acids⁽¹⁾ | 2-15% |

⁽¹⁾ferrulic acid + p-courmaric acid + protocatechic acid + caffeic acid

Thus, according to one variant embodiment of the invention in terms of its first and second objects, the polyphenol compound derived from pine bark originates from a maritime pine extract. Such a maritime pine extract is in particular described in the article “A review of the French Maritime Pine Bark Extract (PYCNOGENOL®), a herbal medication with a diverse clinical pharmacology”, P. ROHDEWALD, International Journal of Clinical Pharmacology and Therapeutics, Vol. 40-No 4/2002(158-168).

According to one preferred embodiment of the invention, in particular in the context of the implementation of the third subject of the invention, the polyphenol compound is a catechin polyphenol as defined hereinafter, and according to one most particularly preferred embodiment, it is a polyphenol compound derived from pine bark.

The catechin polyphenol subgroup comprises a collection of compounds conventionally isolated from plants such as cocoa, tea, grapevine and its derivatives, pine (Pinus maritima), cachou or certain fruits, and having a varying degree of polymerization.

The base unit, also known as catechin or catechol, is 3,5,7,3′,4′-penta-hydroxy-2,3-dihydro-2-phenylchromene, which may be in cis or trans form; epicatechin is its isomer, and may also be present in cis or trans form.

The catechin polyphenols encompass equally the various isoforms of the base units (monomers) and oligomers (or proanthocyanidols) or polymers (tannins).

More particularly, the catechin polyphenols that are of use according to the invention are chosen from the group comprising: catechin, epicatechin, gallocatechin and epigallocatechin, and salts thereof, esters thereof and/or derivatives thereof in the form of monomers or oligomers.

When oligomers are used, they advantageously comprise from 2 to 14 base units, in particular from 2 to 10.

Preferably, their degree of polymerization is less than or equal to 5.

Compounds generally known as proanthocyanidols or procyanidols, also known as anthrocyanin precursors or oligomeric proanthocyanins (OPCs) are in particular used. A part of these oligomers will be degraded after oral absorption so as to release the monomers.

These polyphenols can be conjugated with sugars, for instance glucose, galactose, rhamnose or galacturonic acid. The expression “catechin polyphenols of use according to the invention” is intended to mean, in particular in the present text, mixtures in all proportions of monomers and of the various oligomers comprising from 2 to 14 units, as defined above.

Among the widespread dimers of the family of procyanidols or oligomeric proanthocyanidins, and the use of which is particularly advantageous in the context of the present invention, mention may be made of procyanidin B1, procyanidin B2, procyanidin B3 or else procyanidin B6 or B7.

The procyanidins B1, B2 and B3 are present in particular in plant extracts of cocoa, of apple, of blueberry, of horse chestnut, of hops, of guava and of hazel. Thus, in addition to the pine bark extract, the use of one of these plant extracts is also preferred in the context of the implementation in particular of the third subject matter of the present invention, i.e. the cosmetic use of the combination of glucosamine and of at least one polyphenol compound as a mixture of active agents for maintaining and/or restoring the biomechanical properties of the skin.

The composition according to the invention preferably provides the polyphenol compound at a daily dose ranging from 1 to 1000 mg/day, preferably from 10 to 150 mg/day, and even more preferably from 30 to 100 mg/day.

The composition according to the invention preferably comprises the polyphenol compound at a content ranging from 0.0001% to 50% by weight, preferably from 0.05% to 10% by weight, and even more preferably from 0.5% to 2% by weight, relative to the total weight of the composition.

The cosmetic oral and/or parenteral use of the combination of glucosamine and of at least one polyphenol compound as a mixture of active agents, which is the subject of the invention, makes it possible to maintain and/or restore the biomechanical properties of the skin.

The combination of glucosamine and of at least one polyphenol compound is in particular for maintaining and/or restoring the extensibility, tonicity, firmness, suppleness, density and elasticity properties of the skin.

The skin disorders that can be prevented and/or that the combination in accordance with the invention can combat can be induced by chronological ageing and extrinsic ageing, in particular photoageing.

The present invention also relates to the cosmetic oral and/or parenteral use of glucosamine and of at least one polyphenol compound as a mixture of active agents for preventing and/or combating skin disorders induced by weight loss. This weight loss may be observed, as was recalled above, during slimming diets.

The combination of glucosamine and of at least one polyphenol compound also makes it possible, in particular in women suffering from cellulite, to obtain, in addition to an improvement, in skin tonicity, a smoothing out of the nodes of fat.

Thus, the orange peel appearance, observed on the skin, in particular of the thighs, is reduced or even prevented, just like the flaccid skin appearance, commonly observed, in particular on the arms and the abdomen, in individuals in the course of a slimming diet.

This combination of glucosamine and of at least one polyphenol compound may thus be an aid for decongesting the tissues in this context of slimming or weight-loss diets.

Furthermore, this combination makes it possible to combat the visual aspects of cellulite, such as nodes of fat and the “orange peel” appearance. Thus, the present invention also extends to the cosmetic oral and/or parenteral use of the combination of glucosamine and of at least one polyphenol compound for preventing and/or treating the visual aspects associated with cellulite, such as nodes of fat and the “orange peel” appearance.

Finally, the present invention relates to the cosmetic oral and/or parenteral use of glucosamine and of at least one polyphenol compound as a mixture of active agents for promoting cicatrization.

In addition, a subject of the invention is also the use of a combination of glucosamine and of at least one polyphenol compound for the preparation of a composition for oral administration for preventing and/or treating the cutaneous signs of ageing associated with a loss of extensibility, of tonicity, of firmness, of suppleness and/or of elasticity of the skin.

A subject of the invention is also the use of a combination of glucosamine and of at least one polyphenol compound for the preparation of a composition for oral and/or parenteral administration for reducing the pain on palpation induced by cellulite.

As illustrated in Examples 6 and 7 which follow, the inventors have demonstrated that a composition containing glucosamine and a polyphenol compound derived from pine bark can act, on the one hand, favorably on the dermal matrix, by means of an increased activation of cellular metabolism and a targeted action on the fibroblast cytoskeleton, and, on the other hand, on the expression of dermal cutaneous markers associated with the biomechanical properties of the skin, and in particular with skin firmness.

Consequently, a composition containing glucosamine and a polyphenol compound derived from pine bark is particularly suitable for the prevention and/or treatment of disorders associated with a loss of extensibility, tonicity, firmness, suppleness and/or elasticity properties of the skin, in particular induced by chronological ageing, especially of the mature skin of premenopausal or postmenopausal women, but also induced by photoageing.

This composition is therefore also suitable for the prevention and/or treatment of skin disorders induced by the menopause.

Thus, the present invention also relates to the cosmetic use of a composition containing glucosamine and a polyphenol compound derived from pine bark, in accordance with the invention, for the prevention and/or treatment of skin disorders induced by the menopause.

A composition containing glucosamine and a polyphenol compound derived from pine bark is also particularly suitable for the prevention and/or treatment of the abovementioned skin disorders, i.e. associated with a loss in terms of biomechanical properties of the skin, induced by weight loss, as observed during slimming and/or weight loss diets, such as sagging of the supporting tissues, loss of skin tonicity and elasticity, and increased visibility of nodes of fat.

A composition containing glucosamine and a polyphenol compound derived from pine bark is, in addition, suitable for the prevention and/or treatment of the visual aspects associated with cellulite, such as nodes of fat and the “orange peel” appearance, as is described above.

The composition is, finally, suitable for promoting cicatrization. This property follows in particular from the observation, by the inventors, of the favorable action of the dietary supplement in Example 1 on the significant increase in vimentin.

The compositions according to the invention may be cosmetic, dermatological or pharmaceutical compositions.

For the purpose of the present invention, a cosmetic composition denotes a composition capable of producing an effect on the skin from an esthetic and comfort point of view, or else for beauty purposes, for example with a view to protecting it, maintaining it in good condition, modifying the appearance thereof, and especially embellishing it. It may be in the form of a nutritional product.

The compositions in accordance with the invention, depending on whether they are administered orally and/or parenterally, may be in any of the galenical forms normally used in the method of administration concerned.

In the case of oral administration, a composition in accordance with the present invention may be used in a formulation of dietary supplement or functional food product type, or else of pharmaceutical composition type.

Such a composition may in particular be in the form of soft capsules or gelatin capsules, hoop-cased gelatin capsules, gels, dry or liquid emulsions, tablets, powders to be diluted or oral phials, or any other form known to those skilled in the art.

The composition may optionally contain suitable formulation excipients, such as dye, sweetener, filler, binder, preservative, etc.

According to one preferred embodiment, the active ingredients may be incorporated into food matrices with a view to producing functional food products such as food bars, enriched food products such as oils, margarines, compacted powders, or fibers, or else in the form of an emulsion in drinks.

The composition may also contain compounds such as antioxidants, vitamins or minerals authorized in Europe in dietary supplements, as described in EC Directive 2002/46.

In the case of parenteral administration, a composition in accordance with the present invention may be in the form of an injectable solution or of a patch or transdermal delivery system.

According to one advantageous embodiment of the invention, the composition using glucosamine alone or optionally in combination with at least one polyphenol compound also comprises at least one anti-ageing nutritional active agent, one photoprotective nutritional active agent, one menopause nutritional active agent and/or one slimming nutritional active agent.

Among the anti-ageing nutritional active agents, mention may in particular be made of dietary antioxidants, nutrients with free-radical scavenging properties and cofactors of endogenous antioxidant enzymes: vitamins A, C, E, carotenoids such as lycopene, xanthophylls, isoflavones, certain minerals such as zinc, copper, magnesium or selenium, lipoic acid, co-enzyme Q10, superoxide dismutase (SOD) or alternatively taurine. Among the anti-ageing active agents, mention may in particular be made of unsaponifiable fractions extracted from lipids of plant origin, aloe vera, natural or hydrolyzed marine collagen, plant or marine oils rich in omega-3 fatty acids, in omega-6 fatty acids (including gamma-linolenic acid), etc.

Among the photoprotective nutritional active agents, mention may in particular be made of: antioxidants and free-radical scavengers: vitamins A, C and E, carotenoids, xanthophylls, certain minerals such as zinc, copper, magnesium or selenium, co-enzyme Q10, superoxide dismultase (SOD), and probiotics.

Mention may also be made of nutritional ingredients having hydrating or else immunomodulating properties: probiotics, extract of Polypodium leucotomos, plant or marine oils rich in Ω-3 fatty acids, in Ω-6 fatty acids, including gamma-linolenic acid.

In the context of the present invention, when the glucosamine is combined with at least one polyphenol compound derived from pine bark, the composition comprising this combination may also comprise polyphenol compounds other than the polyphenol compound derived from pine bark, as an addition.

Among the nutritional active agents that are active on the clinical signs of the menopause (for example, hot flushes, etc.), mention may in particular be made of isoflavones, lignans, DHEA, extract of yam, of sage or of hops, calcium, magnesium, protein hydrolysates, and plant or marine oils rich in omega-3 fatty acids.

Among the nutritional ingredients used in the slimming field, mention may in particular be made of: green tea, in particular in extract form, white tea, black tea, grey tea, rooibos (also known as red tea), maté, common horse chestnut, cola, caffeine, theobromine, synephrine, bromelain, ephedra, Citrus aurantium, calcium, hoodia, garcinia, chitosan, plant fibers (cactus, apples, pineapple, etc.), fennel, blackcurrant, meadowsweet and black radish.

Extract of green tea is particularly advantageous by virtue of its natural content of flavonoids and more particularly of catechins and epigallocatechin gallate (EGCG), concerning in particular antioxidant properties.

According to one particular embodiment, the invention relates to a composition for oral administration comprising the combination of glucosamine, of at least one polyphenol compound derived from pine bark and of an extract of green tea. According to one even more particular embodiment, this same composition also comprises calcium, for example in the form of calcium carbonate, calcium of marine origin, calcium lactate or calcium citrate.

According to another embodiment, the present invention also relates to the cosmetic oral and/or parenteral use of the combination of glucosamine, of at least one polyphenol compound and also, optionally, of calcium, as a mixture of active agents for maintaining and/or restoring the biomechanical properties of the skin and/or for preventing and/or treating skin disorders induced by cellulite.

According to these two embodiments, the green tea may be present in the composition at a content ranging from 50 mg to 3 g, in particular from 300 to 800 mg, this composition preferably being administered at a rate of one dose per day.

Still according to these two embodiments, the calcium may be present in the composition at a content ranging from 300 mg to 2 g, preferably from 300 mg to 1 g, preferably at a rate of one dose per day.

The dietary supplement in accordance with the present invention, comprising glucosamine in a first composition and at least one polyphenol compound derived from pine bark in a second composition, as a kit or combination product for simultaneous, separate or sequential use, can be formulated in such a way that the two compositions are in the same forms or in different forms, for example chosen from those mentioned above.

Such a kit may in particular be provided in one and the same packaging or in two distinct packagings, one for each composition.

The examples which follow illustrate the present invention.

### EXAMPLE 1

### Effect of Glucosamine on Collagen Synthesis and the Expression of CD44-in vitro Skin Explant Model

The aim of this study was to demonstrate the effect of glucosamine (in sulfated form) on an epidermal marker (CD44), potentially involved in the pathogenesis of cutaneous atrophy, a major manifestation of cutaneous ageing, and on collagen synthesis.

For this, a method of culturing was used which allows human skin to be kept alive under metabolic conditions close to in vivo (individuals aged between 50 and 60).

The glucosamine in aqueous solution was added to the culture medium, at the plasma concentration (5 μM), thus making it possible to simulate oral and/or parenteral administration.

At the epithelial level, the epidermal hyaluronic acid receptor was examined (anti-CD44 antibody). At the dermal level, collagen neosynthesis by fibroblasts (by chemical assay) was evaluated.

Materials and Methods

Keeping Human Skin Alive

Skin fragments were obtained after plastic surgery (mammary or abdominal plasties) from menopausal women (8 different donors from female individuals aged between 50 and 60). The fragments are placed in inserts which are themselves suspended above culture wells. Culture medium is added to the bottom of the wells, the medium passing by slow diffusion between the two compartments by means of a porous membrane (12 μm).

From D0 to D10, glucosamine was added to the culture medium of the skin fragments, every day. For the CD44 analysis, a series of skin samples was taken at D4. A second series was taken at D10 for assaying the collagen synthesis.

Analyses

Immunohistochemical Demonstration of the Hyaluronic Acid Receptor in the Epithelium (CD44)

It is possible to demonstrate a transmembrane glycoprotein of 80-95 kD, CD44 (H-CAM, Novocastra, dilution to 1/300), the hyaluronic acid receptor, in the epithelial. The immunodetection was carried out using the CSA kit (DAKO) and visualized in red with AEC (3-amino-9-ethylcarbazole).

Semi-quantitative scores made it possible to specify the intensity of the immunolabeling (scores 0 to 4, from negative to intense). The topography was also specified by means of scores:
score of 0: no labeling
score 1: labeling of basal layer,
score 2: labeling of the basal layer and of a third of the epithelium,
score 3: labeling of the basal layer and of two thirds of the stratum mucosum,
score 4: labeling of the entire epithelium.

Biochemical Assaying of Collagen

After the 10 days of being kept alive, the skin fragments are digested enzymatically overnight at +4° C. in a 0.5M acetic acid solution containing pepsin. This method makes it possible to recover the newly synthesized collagen. After grinding using a Potter, the amount of collagen (μg/ml) is evaluated using a spectrocolorimetric assaying method at 540 nm: the acid-soluble collagen is detected after specific binding of the dye Sirius red (Sircol Collagen Assay, Interchim).

In order to compare the various results, the amount of collagen is related to the amount of total proteins of the sample. The protein concentration assay was carried out spectrophotometrically at 562 nm (BCA assay, Pierce). The final result is expressed in μg of collagen/mg of protein.

Results

Immunohistochemical Demonstration of the Hyaluronic Acid Receptor in the Epithelium (CD44)

The results are expressed in Table I. In the presence of glucosamine, a significant increase in intensity of the CD44 labeling is noted, with a score of 3.4 versus 2.7 for the control skin (p=0.02).

TABLE 1
Immunohistochemical analysis of the epithelial hyaluronic acid
receptor, CD44 (mean semi-quantitative scores for labeling
topography and intensity)

| | Intensity | Topography |
|---|---|---|
| | score | score |
| Control skin | 2.7 ± 1.1 | 3.4 ± 0.7 |
| Skin + glucosamine (5 μM) | 3.4 ± 0.7 | 3.7 ± 0.5 |
| | *p = 0.02 | p = 0.054 |

*statistically significant difference compared with the control skin (paired Student's test, p < 0.05)

Biochemical Assaying of Collagen

The collagen synthesis is significantly increased in the presence of glucosamine: 282 μg/ml (p=0.031) in comparison with the control skin, where the rate is 177.5 μg/ml.

Conclusion

Under the test conditions, glucosamine administered at 5 μM significantly stimulates i) collagen synthesis by dermal fibroblasts and also 2) expression of the CD44 hyaluronic acid receptor. Glucosamine therefore appears to be a fibroblast metabolism activator and an agent for promoting the reestablishment of epidermal homeostasis, these being parameters which are generally impaired with age.

### EXAMPLE 2

### Evaluation of the Anti-Degradation Activity of glucosamine

The objective of these tests is to evaluate the effect of glucosamine sulfate on the activity against degradation of the dermal macromolecules (anti-MMP activity) and on their contractile properties (action of the cytoskeleton). The effect on the mechanical properties of the fibroblasts is evaluated by measuring the speed of retraction of a three-dimensional collagen gel.

Materials and Methods

Fibroblast Cultures

Human fibroblasts, harvested by growth from skin explants from a normal young donor, are routinely cultured in DMEM containing 10% of fetal bovine serum (FCS), ascorbic acid (50 μg/ml), penicillin-streptomycin (100 U/ml), referred to in the rest of the report as DMEM-FCS. They are amplified in culture by trypsinizing subconfluent cultures and dividing the dishes 1 in 3. The cultures are used up until passage 14. They are regularly tested for the absence of mycoplasma.

Preparation of Solutions and Sterilization

The glucosamine sulfate is provided in the form of (C₆H₁₄NO₅)₂ S04.2KCl (glucosamine.SO₄ titer: 74%) brand Bioiberica, code F0379, Batch 4/0001. A stock solution at 13.6 mg/ml of distilled water is sterilized by filtration through an Acrodisc, and conserved at −20° C.

2.1. Cytotoxicity

The fibroblasts are seeded into 24-well multiwells at 25 or 50×10³ cells/well in DMEM-FCS. After 6 h of attachment, 20 μl of gradual dilutions of the active agents to be tested are added to the cultures. After 24 h of contact, the medium and the active agents are renewed. After 48 h of culture, the medium is removed, and the cell layers are rinsed twice with physiological saline. The number of cells is determined by measuring DNA by the fluorimetric technique using the bisbenzimide reagent in a “Gemini” fluorimeter. The cultures are prepared in triplicate and the measurements are carried out on each culture in duplicate. The control cultures receive the solvent alone.

Glucosamine Sulfate

The toxicity of glucosamine sulfate is tested at 0.5, 1, 5, 10, 20 and 40× the plasma concentration, i.e. 3.4, 6.8, 34, 68, 136 and 272 μg/ml of culture medium.

| [x × plasma | | | |
|---|---|---|---|
| concentration] | μg/ml | N | DNA (μg/ml) |
| 0 | 0 | 6 | 2.37 ± 0.33 |
| 0.5x | 3.4 | 3 | 2.81 ± 0.41 |
| 1x | 6.8 | 3 | 2.56 ± 0.37 |
| 5x | 34 | 3 | 2.89 ± 0.23 |
| 10x | 68 | 3 | 2.61 ± 0.26 |
| 20x | 136 | 3 | 2.52 ± 0.18 |
| 40x | 272 | 3 | 2.50 ± 0.32 |

x = plasma concentration

No cytotoxic effect is observed for the glucosamine sulfate, even at concentrations representing 40× the plasma concentration. An increase in the number of cells (p=0.04, Student's test) is observed at the concentration of 34 μg/ml, indicating a slight stimulating effect on the rate of proliferation of the fibroblasts.

2.2. Effect of Glucosamine Sulfate on the Activity of MMPs (mRNA—Model of Human dermal Fibroblasts in a Monolayer on Plastic)

1. Preparation of Cultures and Purification of Total RNA

The fibroblasts are seeded in DMEM-FCS in a proportion of 2.10⁵ cells/disk of 6 cm. After 18 h of attachment, the culture medium is renewed with DMEM-FCS containing:

a) glucosamine sulfate alone
[plasma concentration] 2×(n=2)
   5× (n=2)
   10× (n=2)
control (n=3)

2. Measurement of Reference RNAs (28S and GAPDH) and of Specific Messenger RNAs by RT-PCR

The total amount of RNA present in the solution that will be used to carry out the specific mRNA measurements is determined by measuring the 28S ribosomal RNA. The RNA solution is aliquoted and frozen at −80° C. until the specific mRNAs are assayed by RT-PCR. The mRNA results are expressed in arbitrary units (AU) per unit of 28S. The measurements are carried out in triplicate on two or three separate cultures.
MMP3: stromelysin 1 responsible for the degradation of proteoglycans and of other extracellular matrix constituents and activator of MMP 1

| | [plasma conc] | UA/28S |
|---|---|---|
| glucosamine | 0 | 1745 ± 31 |
| | 2x | 1261 ± 120 |
| | 5x | 1162 ± 22 |
| | 10x | 1060 ± 375 |

A significant reduction in the level of MMP3 mRNA is induced by glucosamine sulfate (p<0.005).

Conclusion: Glucosamine reduces the level of MMP3 expression. This indicates an activity against degradation of the dermal matrix constituents, in particular the proteoglycans and glycosaminoglycans. These results demonstrate the glucosamine is an agent capable of protecting the macromolecules of the dermis, the impairment of which contributes to the loss of skin firmness or alternatively to the worsening of skin disorders induced by cellulite.

### EXAMPLE 3

### Effect of Glucosamine Sulfate on the Contractile Properties of Fibroblasts

A sterile solution of purified native collagen is mixed with a suspension of fibroblasts, DMEM-FCS medium and the test products in bacteriological dishes. The mixture is brought to 37° C., this resulting in polymerization of the collagen and the formation of a three-dimensional collagen gel trapping the fibroblasts and floating in the culture medium. The cells attached to the collagen fibers and, under the contractile activity of the cytoskeleton of the fibroblasts, the gel is gradually retracted. This gel is commonly referred to as “Retracting Collagen Gel” or RCG. The article Charles A. Lambert et al. “An Interleukin-1 loop is induced in human skin fibroblasts upon stress relaxation in a three-dimensional collagen gel but is not involved in the up-regulation of Matrix Metalloproteinase 1”, The Journal of Biological Chemistry, Vol. 273, No. 36, pp. 23143-23149, 1998, describes the use of such a collagen gel.

a—A first series of gels containing 25 000 fibroblasts was prepared. The glucosamine was tested at the plasma concentration in triplicate. The control cultures received the solvent alone. The gel diameter measurements show a reduction in diameter of 21 mm in 18 hours for the controls and of 23 mm for the same period of time for the glucosamine. This slight difference reduces as a function of time, up to 50 hours of culture.

b—These results prompted the inventors to reperform the same type of experiment, with glucosamine sulfate alone at 2×, 5× and 10× the plasma concentration, slightly reducing the number of fibroblasts (20 000) in order to slow down the process. The speed of retraction is indeed slowed since a reduction in the diameter of the gel of 19 mm is observed in 46 h. Once again, a slightly greater retraction (22 mm in 46 h) is observed with the three glucosamine concentrations tested.

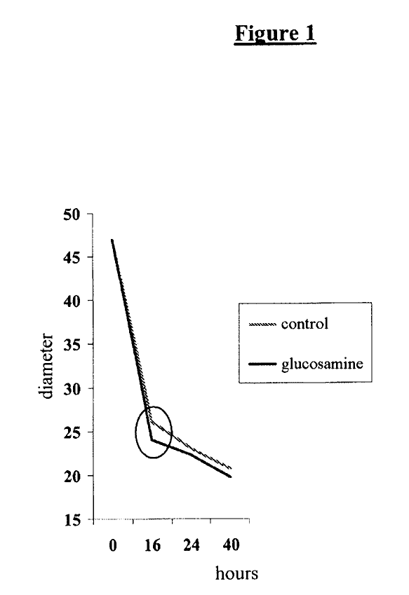

Conclusion: FIG. 1 illustrates the retraction, and therefore the stimulation of the contractile properties, of the fibroblast, which is observed in the presence of glucosamine sulfate. These results demonstrate the activity of glucosamine on the stimulation of the cytoskeleton and the metabolism of the fibroblast and that it is, as a result, an agent capable of acting positively on skin firmness and also on skin disorders induced by cellulite.

### EXAMPLE 4

### Effect of Glucosamine Sulfate on the Expression of Dermal Cutaneous Markers Associated with Skin Firmness

1. Protocol

Restrictive randomized monocentric double-blind study versus placebo. Fifteen women, aged 50 to 65, were given, as a supplement, for 8 weeks, either a placebo (n=7) or glucosamine sulfate (n=8) equivalent to 250 mg of glucosamine (GLU).

A skin biopsy 3 mm in diameter (inner face of the arm) was taken before the beginning (T0) and at the end of the 8 weeks of supplementation (T8).

The total RNA contained in the skin samples was extracted and purified by means of an Ambion Ribo Pure Kit.

The analysis of the specific dermal markers was carried out by RT-PCR as previously described (Nusgens et al, JID, 116: 853-859, 2001). All the results are expressed in arbitrary units relative to the amount of 28S rRNA.

At T0, the homogeneity of the values of each mRNA, between the placebo group and the GLUT group, was verified (ANOVA test and Tukey multiple comparison test).

The effects of the supplementation (placebo or GLU) were then analyzed by comparing, for each volunteer, the individual expression of the mRNAs at T8 and at T0. A T8−T0 ratio was thus calculated. The means of each ratio of the two groups were compared using a Student's test for paired samples.

2. Results

Table 2 summarizes the individual means of the mRNA levels before (T0) and after 8 weeks of supplementation (T8) in the placebo group and in the GLU group.

TABLE 2

| | PLACEBO | GLU |
|---|---|---|
| mRNA | (T8/T0) | (T8/T0) |
| Vimentin | 1.27 ± 0.33 | 1.28 ± 0.33 |
| Decorin | 1.11 ± 0.22 | 1.11 ± 0.11 |
| Fibromodulin | 1.12 ± 0.16 | 1.10 ± 0.12 |
| Biglycan | 1.10 ± 0.18 | 1.15 ± 0.19 |
| Hyaluron synthase | 1.51 ± 0.73 | 1.62 ± 1.42 |

It is noted that the results obtained with the placebo are nonsignificant, whereas the results obtained with glucosamine are significant.

3. Conclusion

Glucosamine (GLU) significantly increases the expression:
of vimentin, a constituent of the cytoskeleton,
of decorin, of fibromodulin and of biglycan, belonging to the SLRP (small leucine rich protein) family. These markers are involved in the architectural organization of the structures of the skin,
of hyaluron synthase, an enzyme involved in the synthesis of hyaluronic acid, the hydrating properties of which are known to those skilled in the art.

The placebo has no effect on these various markers.

These results show that glucosamine has a beneficial effect on the expression of specific genes encoding structural macromolecules of the dermis. This effect is found to be particularly beneficial for maintaining and/or for restoring skin firmness and preventing and/or treating cellulite, two phenomenon characterized by a detrimental alteration of the dermal architecture.

### EXAMPLE 5

### Exploratory Clinical Study Relating to the Quantification of Target mRNAs Induced by Taking Dietary Supplement A1

Formula of Dietary Supplement A1

Dietary supplement A1 is preferably intended for individuals who are on a slimming diet and who are confronted with a loss of skin firmness subsequent to weight loss. Green tea and calcium are ingredients that are respectively recognized as an adjuvant in slimming diets or involved in lipid metabolism.
Galenic from: sachets
Dosage: 1 sachet/day

TABLE 3
Formula dietary supplement A1

| | Usual name of the | | Composition |
|---|---|---|---|
| | ingredient/excipient | Origin/code | (mg/sachet) |
| NUTRITIONAL | Pine bark extract | FLAVOPIN ® | 40.00 |
| INGREDIENTS | | inneov | |
| | Extract of green tea | Givaudan | 375.00 |
| | Calcium carbonate | Rettenmaier | 1000.00 |
| | Glucosamine | Bioiberica | 250.00* |
| | sulfate.2KCl | | |
| EXCIPIENTS | Polyvinylpyrrolidone | E1201 | 20-30 |
| | Sodium saccharinate | E954 | 30-40 |
| | Magnesium stearate | E470b | 1-10 |
| | Lychee flavoring | — | 35-50 |

*glucosamine sulfate equivalent

Objective of the Clinical Trial

To evaluate the effect of dietary supplement A1, versus placebo, on cutaneous biomarkers that may be associated with a change in the biomechanical properties of the skin, after taking dietary supplement A1 for 2 months.

Methodology
2 months study on 16 women menopausal for more than two years (7 in the group with dietary supplement A1/9 in the placebo group), over the age of 50, not undergoing hormone replacement therapy, and exhibiting a lack of skin firmness on the inner face of the arm.
3 mm biopsies are taken from the arm at T0 and T2 months for extraction of total mRNA.
Specific mRNAs encoding proteins that may be associated with a change in the biomechanical properties of the skin are quantified according to the following protocol.

Preparation of mRNAs for the Purpose of RT-PCR

The two biopsies were combined and ground in liquid nitrogen (Mikro Dismenbrator S, B. Braun Biotech International), and than the total RNA was extracted and purified by cesium chloride gradient ultracentrifugation. The amount of RNA purified was evaluated by measuring the OD (optical density) at 260 nm (nanodrop) and the quality of the extracted RNA was validated by calculating the OD260/OD280 ratio. The integrity of the extracted RNAs was also verified using the BioAnalyzer from Agilent. Stock solutions of RNA were prepared in order to obtain a concentration close to 1.25 ng/μl.

Evaluation of the Amount of Cutaneous Biomarkers

The RNA concentration was standardized relative to the amount of 28S ribosomal RNA. The RT-PCR technique was made quantitative by adding, to each reaction tube, an internal standard of known concentration and consisting of a synthetic RNA, which will be cotranscribed and coamplified at the same time as the RNA being sought. The samples corresponding to the T0 and to the T2 of each volunteer were analyzed in the same series, and were loaded onto and migrated on the same polyacrylamide gel. The statistical analysis was carried out by means of a one-sided Student's test for paired samples, by comparing the values at T0 and at T2, in each group. A ratio T2/T0=1.00 signifies that the two values are comparable. A ratio T2/T0>1.00 reflects an increase in the transcript studied. Conversely, a ratio of T2/T0<1.00 reflects a decrease in the expression of the mRNA studied.

Results/Quantification of Target mRNAs

TABLE 4
Target gene expression after taking dietary supplement A1
(versus placebo)

| | |
|---|---|
| | T2/T0 |
| | DIETARY |
| T2/T0 | SUPPLEMENT |

| | Placebo | paired | A1 | paired |
|---|---|---|---|---|
| | (n = 9) | t test | (n = 7) | t test |
| VIM | 1.02 + 0.36 | NS | 1.43 + 0.47 | 0.025 |
| BMP1 | 1.14 ± 0.53 | NS | 1.55 ± 0.67 | 0.03 |
| DEC | 1.33 ± 0.58 | NS | 1.59 ± 0.53 | 0.02 |
| LUM | 1.25 ± 0.53 | NS | 1.69 ± 0.55 | 0.004 |
| FIBMOD | 1.17 ± 0.44 | NS | 1.93 ± 0.77 | 0.03 |
| ACTIN | 0.89 ± 0.32 | NS | 1.49 ± 0.48 | 0.005 |

VIM: vimentin;
BMP1: Bone Morphogenetic Protein 1;
DEC: decorin;
LUM: lumican;
FIBMOD: fibromodulin;
ACTIN: actin.

The concentration of mRNA encoding vimentin is significantly increased (p=0.025) after taking dietary supplement A1, whereas there is no significant difference with the placebo. Vimentin is a protein which is part of the cytoskeleton of the cells of the dermis. Taking dietary supplement A1 for 2 months leads to a statistically significant increase in the mRNA encoding actin (p=0.0005), another protein constituting the cytoskeleton. The placebo has no effect on any marker.

The term “cytoskeleton” denotes the network of dynamic intracellular fibers involved in all the movements of the cell, for instance the intracellular transport of the organelles or maintaining the shape of the cell. The elements of the cytoskeleton decrease with age, inducing a slowing down of the synthetic metabolism of the cell.

In cells of mesenchymal origin, such as fibroblasts and endothelial cells, vimentin is a major structural compound of the intermediate filaments. This cytoskeletal network is directly involved in the mechanical cellular functions. In particular a low expression of vimentin affects cicatrization processes. Vimentin-deficient cells exhibit in particular a weak mechanical stability and also a reduced motility and reduced contractile properties. Furthermore, vimentin participates in the spatial organization of focal complexes, in the organization of actin-dependent microfilaments and also in the interactions with the extracellular matrix. Some of these interactions are directly affected by cutaneous ageing, like those which have an effect on the control of cell migration, proliferation and contraction or else on the control of the metabolic phenotype.

Actin is a cytoskeletal protein which participates in the formation of actomyosin stress fibers and cortical actin polymers which are involved in the mechanical functions of cells within the cytoskeleton.

The increase in mRNAs encoding actin and vimentin therefore indicates that dietary supplement A1 acts favorably on the cytoskeleton of fibroblasts. It is the sign of an increased activity of the cell.
the decorin gene is significantly (p=0.02) overexpressed following the taking of dietary supplement A1, whereas the expression of this gene is not modified following the taking of the placebo. Decorin, which belongs to the Small Leucine-Rich Proteoglycan (SLRP) family, is a small glycosylated protein (proteoglycan). Decorin is present in the entire dermis, but absent from the epidermis. It is synthesized and secreted by fibroblasts. In addition, after the supplement has been taken, a significant increase in the genes encoding other Small Leucine-Rich Proteoglycans (SLRPs), in particular those of lumican (p=0.004) and of fibromodulin (p=0.03), is also noted. These markers are involved in the architectural organization of the structures of the skin.

Finally, taking dietary supplement A1 leads to a statistically significant increase in the expression of the mRNA encoding BMP-1 (Bone Morphogenetic Protein) (p=0.03), whereas this effect is not found with the placebo. BMP-1 is an enzyme which cleaves procollagens I and III, thus forming mature monomers capable of assembling within the collagen fibrils. Through the combined action of the BMP-1 enzyme and of ADAMTS-2, the procollagen propeptides are cleaved so as to allow them to subsequently self-polymerize so as to form collagen fibers and fiber bundles that are mechanically stronger. The size of these fibers and also the regularity thereof are controlled by decorin, lumican and fibromodulin. In addition, the BMP-1 enzyme is directly involved in the conversion of probiglycan to biglycan.

The results on the SLRPs and BMP-1 show that dietary supplement A1 appears to have a stabilizing effect on the structure of collagen fibers and therefore contributes to improving the quality of the extracellular matrix of the dermis.

By virtue of these two results, on the one hand the action on the cytoskeleton revealed by the study of vimentin and actin, and, on the other hand, the action on collagen fibers revealed by the study of decorin and BMP-1, dietary supplement A1 exhibits an overall and complementary action on the dermis.

### EXAMPLE 6

### Clinical Study Aimed at Evaluating the Effect of Dietary Supplement A1 on Skin Properties in Overweight Women Who are Following a Slimming Diet

Formula of Dietary Supplement A1

Dietary supplement A1 is preferably intended for individuals who are following a slimming diet and who are confronted with a loss of skin firmness subsequent to weight loss. Green tea and calcium are ingredients respectively recognized as an adjuvant in slimming diets or involved in lipid metabolism.
Galenical form: sachets
Dosage: 1 sachet/day

Objective of the Clinical Trial

The principal objective of this study is to evaluate the effect of dietary supplement A1 on skin properties in the course of a slimming diet in women, on the basis of data obtained with the clinical score and self-evaluation by the volunteers.

Methodology

Clinical Phase
3-month study on 40 women (placebo n=19, A1 n=21), aged 25 to 45, monitored by a nutritionist, in a private clinic, in the context of a slimming diet. This study was carried out versus placebo.
The clinical score is evaluated by the dermatologist at T0 and T3 months. This clinical score is the result of the sum of the scores for laxity, withering, wizened appearance and ptosis, evaluated on a scale of 0 (no) to 6 (considerable). This score reflects the tonicity of the skin.
The self-evaluations were carried out for each patient at T0 and at T3 months using a scale ranging from 0 (none) to 9 (very large).

Results

Clinical score: after 3 months, the decrease in the clinical score is statistically greater in the A1 group than in the placebo group (p=0.04). It should be noted that the clinical scores are comparable at T0. This result favors an improvement in the biomechanical properties of the skin following the taking of dietary supplement A1.

Self-evaluation: the scoring carried out by the volunteers demonstrates, after A1 has been taken for 12 weeks, statistically significant changes:
Decrease in the orange peel appearance on the thigh, on palpation (p=0.004) and after observation (p=0.001).
Decrease in the flaccid skin appearance on the arm (p=0.04) and on the abdomen (p=0.04).
Decrease in pain in response to pinching on the thigh (p=0.05).

These two results demonstrate a beneficial effect of dietary supplement A1 on the skin properties, with an improvement in skin tonicity and smoothing out of fat nodes, a characteristic sign of cellulite.

### EXAMPLE 7

### Effect of Glucosamine Sulfate+Polyphenols (Maritime Pine Bark Extract) on the Expression of Dermal Cutaneous Markers Associated with the Biomechanical Properties of the Skin

1. Protocol

Restrictive randomized monocentric double-blind study versus placebo. 15 women, aged 50 to 65, were given, as a supplement, for 8 weeks, either a placebo (n=7) or a mixture of glucosamine sulfate (equivalent to 250 mg of glucosamine) and of maritime pine bark extract (30 mg; n=8, GLU 02).

A skin biopsy 3 mm in diameter (inner face of the arm) was taken before the beginning (T0) and at the end of the 8 weeks of supplementation (T8).

The total RNA contained in the skin samples was extracted and purified by means of an Ambion Ribo Pure kit.

The analysis of the specific dermal markers was carried out by RT-PCR as previously described (Nusgens et al, JID, 116: 853-859, 2001). All the results are expressed in arbitrary units relative to the amount of 28S rRNA.

At T0, the homogeneity of the values of each mRNA, between the placebo group and the GLUT group, was verified (ANOVA test and Tukey multiple comparison test).

The effects of the supplementation (placebo or GLU 02) were then analyzed by comparing, for each volunteer, the individual expression of the mRNAs at T8 and at T0. A T8/T0 ratio was thus calculated. The means of each ratio of the two groups were compared using a Student's test for paired samples.

2. Results

Table 5 summarizes the individual means of the amounts of mRNA before (T0) and after 8 weeks of supplementation (T8) in the placebo group and in the GLU 02 group.

TABLE 5

| | PLACEBO | |
|---|---|---|
| mRNA | (T8/T0) | GLU (T8/T0) |
| Vimentin | 1.27 ± 0.33 | 1.36 ± 0.27 |
| Collagen A1 I | 1.48 ± 0.51 | 2.00 ± 0.94 |
| Collagen A1 III | 1.38 ± 0.41 | 1.94 ± 0.74 |
| Decorin | 1.11 ± 0.22 | 1.23 ± 0.16 |
| Lumican | 1.06 ± 0.23 | 1.12 ± 0.12 |
| Fibromodulin | 1.12 ± 0.16 | 1.21 ± 0.18 |
| Biglycan | 1.10 ± 0.18 | 1.22 ± 0.17 |
| Actin | 1.10 ± 0.15 | 1.16 ± 0.23 |

It is noted that the results obtained with the placebo are nonsignificant, whereas the results obtained with the glucosamine+polyphenols combination are significant.

3. Conclusion

The glucosamine+polyphenol combination significantly increases the expression:
of collagens type I and III, which are major constituents of the dermal architecture,
of vimentin and of actin, which are constituents of the cytoskeleton,
of decorin, of fibromodulin, of lumican and of biglycan, which are all members of the SLRPs (Small Leucine Rich Protein) family. These markers are involved in the architectural organization of the structures of the skin.

The placebo has no effect on these various markers.

These results show that the combination of glucosamine with a polyphenol compound extracted from maritime pine bark has a beneficial effect on the expression of specific genes encoding structural macromolecules of the dermis. This effect is found to be particularly beneficial for maintaining and/or restoring the biomechanical properties of the skin, including for maintaining and/or restoring skin firmness and preventing and/or treating cellulite, two phenomenon characterized by a detrimental alteration of the dermal architecture.

### EXAMPLE 8

### Gel Capsule Form (Fish Gelatin)

| Ingredient/additive | Dosage (mg/capsule) |
|---|---|
| Glucosamine sulfate.2KCl | 200 |
| Microcrystalline cellulose | 50 |

### EXAMPLE 9

### Drink Form

| Ingredient/additive | Dosage (mg/75 ml) |
|---|---|
| Glucosamine sulfate.2KCl | 1000 |
| Water | qs100 |

### EXAMPLE 10

### Dilutable Powder Form (Sachet)

| | Dosage |
|---|---|
| Ingredient/additive | (mg/sachet) |
| Pine bark extract | 20.0 |
| Glucosamine sulfate.2KCl | 170.0 |
| Extract of green tea | 187.5 |
| Calcium carbonate | 500.0 |
| Modified starch | 500 |
| Colloidal silica | 32.0 |
| Aspartame | 35.0 |
| Allura red | 11.2 |

The posology is 2 sachets per day.

### EXAMPLE 11

### Capsule Form (Fish Gelatin)

| | Dosage |
|---|---|
| Ingredient/additive | (mg/capsule) |
| Pine bark extract | 10.0 |
| Glucosamine sulfate•2KCl | 85.0 |
| Extract of green tea | 93.7 |
| Calcium carbonate | 250.0 |
| Magnesium stearate | 10.0 |
| Microcrystalline cellulose | 46.2 |
| Sodium carboxymethylcellulose | 5.0 |

The posology is 2 to 4 gel capsules per day.

1. A method for preventing or treating skin disorders induced by cellulite, or for maintaining or restoring skin firmness, comprising the oral or parenteral use of glucosamine as an active agent by a person in need thereof.

2. The method according to claim 1, wherein the method is for restoring skin firmness, wherein the loss of said skin firmness is induced by chronological ageing.

3. The in method according to claim 1, wherein the method is for restoring skin firmness, wherein the loss of said skin firmness is induced by hormonal ageing.

4. The method according to claim 1, wherein the method is for restoring skin firmness, wherein the loss of said skin firmness is induced by extrinsic ageing.

5. The method according to claim 1, wherein the method is for restoring skin firmness, wherein the loss of said skin firmness is induced by weight loss.

6. The method according to claim 1, wherein the method is for preventing or combating cutaneous pain or pinching induced by cellulite.

7. The method according to claim 1, wherein the method is for preventing or treating the visual aspects associated with cellulite.

8. The method according to claim 1, wherein the glucosamine acts as at least one of an activator of collagen synthesis, an agent for promoting the reestablishment of epidermal homeostasis, an agent for inhibiting the expression of MMP3, an agent for promoting fibroblast contractility, and an agent for stimulating the expression of cutaneous markers.

9. A method for preventing or treating skin disorders induced by the menopause, comprising the oral or parenteral use of glucosamine, as an active agent.

10. The method according to claim 1, wherein the glucosamine is in combination with a polyphenol compound.

11. The method according to claim 10, wherein the polyphenol compound is derived from plant extracts of green tea, grapes pine apple, blueberry, hops, guava, cocoa, or wood.

12. The in method according to claim 10, wherein the polyphenol compound is in the form of proanthocyanidins, flavonoids, lignans, lignins, stilbenes, coumarins, or a combination thereof.

13. The method according to claim 12, wherein the polyphenol compound is a catechin polyphenol comprising catechin, epicatechin, gallactocatechin, or epigallactocatechin, in the form of monomers or oligomers.

14. (canceled)

15. A method for maintaining or restoring the biomechanical properties of the skin, or for preventing or treating skin disorders induced by cellulite, comprising the oral use of the combination of glucosamine and of at least one polyphenol compound as a mixture of active agents by a person in need thereof.

16. The method according to claim 15, wherein the method is for maintaining or restoring the extensibility, tonicity, firmness, suppleness, density or elasticity properties of the skin.

17. The method according to claim 15, wherein the method is for preventing or combating skin disorders induced by chronological ageing.

18. The method according to claim 15, wherein the method is for preventing or combating skin disorders induced by extrinsic ageing.

19. The method according to claim 15, wherein the method is for preventing or combating skin disorders induced by weight loss.

20. A method for promoting cicatrization comprising the oral or parenteral use of the a combination of glucosamine and of at least one polyphenol compound as a mixture of active agents.

21. The method according to claim 1, wherein the glucosamine is in at least one of the form of a salt, acetylated form, and polymeric form.

22. The method according to claim 1, wherein the glucosamine is in the form of a salt selected from the group consisting of glucosamine sulfate, glucosamine sulfate potassium chloride, glucosamine sulfate sodium chloride and glucosamine hydrochloride.

23. A method for promoting the reestablishment of epidermal homeostasis, for limiting the degradation of dermal matrix constituents, for reducing the level of MMP3 expression, for stimulating the fibroblast cytoskeleton, for improving the contractile properties of fibroblasts, or for increasing the expression of cutaneous markers, reducing cellulite and also the associated visual aspects, or maintaining or restoring skin firmness, wherein said method comprises the oral or parenteral administration of a composition comprising glucosamine optionally in combination with a polyphenol compound.

24. A composition for oral administration comprising a combination of glucosamine and of at least one polyphenol compound derived from pine bark.

25. The composition according to claim 24, wherein the glucosamine is present in at least one of the form of a salt, acetylated form, and polymeric form.

26. The composition according to claim 24, wherein the polyphenol compound derived from pine bark has aphenolic trimer content that can range from 5% to 25% by weight, relative to the total weight of the polyphenol compound.

27. The composition according to claim 24, wherein the polyphenol compound derived from pine bark has a polyphenolic dimer content that can range from 5% to 25% by weight, relative to the total weight of the polyphenol compound.

28. The composition according to claim 24, wherein the polyphenol compound derived from pine bark has from 2% to 15% by weight, of phenolic acids of ferrulic acid, p-coumaric acid, cafeic acid and protocatechic acid type, relative to the total weight of the polyphenol compound.

29. The composition according to claim 24, wherein the glucosamine is present at a content ranging from 0.0001% to 80% by weight relative to the total weight of the composition.

30. (canceled)

31. The composition according to claim 24, comprising at least one of an anti-ageing nutritional active agent, a photoprotective nutritional active agent, a menopause nutritional active agent, and a slimming nutritional active agent.

32. The composition method according to claim 24, wherein it the composition is in the form of soft capsules, hoop-cased gel capsules, gels, dry or liquid emulsions, tablets, powders to be diluted or oral phials, or alternatively a functional food product.

33. A dietary supplement or a functional food product comprising glucosamine in a first composition and at least one polyphenol compound derived from pine bark in a second composition, as a kit or a combination product for simultaneous, separate or sequential use.

34. (canceled)

35. The process according to claim 23, wherein the cutaneous markers are selected from the group consisting of vimentin, decorin, fibromodulin, biglycan and hyaluron synthase.

36. The composition according to claim 24, wherein the glucosamine is in the form of a salt selected from the group consisting of glucosamine sulfate, glucosamine sulfate potassium chloride, glucosamine sulfate sodium chloride and glucosamine hydrochloride.
